(19) United States
(12) Patent Application Publication
(10) Pub. No.: US 2026/0000345 A1
(43) Pub. Date: Jan. 1, 2026

(54) AT-HOME CONTACTLESS FETAL MOVEMENT TRACKING

(71) Applicant: Google LLC, Mountain View, CA (US)

(72) Inventor: Jaime Lien, Mountain View, CA (US)

(73) Assignee: Google LLC, Mountain View, CA (US)

(21) Appl. No.: 18/580,339

(22) PCT Filed: Mar. 22, 2023
(86) PCT No.: PCT/US2023/015942

§ 371 Date: Jan. 18, 2024

(51) Int. Cl. A61B 5/00; A61B 5/05; A61B 5/11

(52) U.S. Cl. CPC A61B 5/4362; A61B 5/05; A61B 5/1114; A61B 5/7264; A61B 5/742; A61B 5/749

(57) ABSTRACT

Various arrangements for performing contactless fetal movement tracking are detailed herein. User input can first be received from an expectant mother requesting that contactless fetal movement monitoring be performed. A state analysis on the expectant mother may be performed to determine that the expectant mother is present and static in a bed at which the contactless fetal movement tracking device is pointed. Fetal movement tracking may be performed using radar data received from a radar sensor of the contactless fetal movement tracking device while the state analysis indicates that the expectant mother is present and static in the bed. A fetal tracking report may then be presented based on the performed fetal movement tracking.

### BACKGROUND

A significant decrease in human fetal movement in the second and third trimester of a pregnancy can be a sign of fetal distress or impairment. Expectant mothers are recommended to count and track the number of kicks or movements felt daily, starting around the 28th week of pregnancy to monitor for any decreases in the amount of fetal movement. Currently, at home, most expectant mothers perform this task manually. This arrangement has significant drawbacks. First, the expectant mother has to remember to perform the monitoring. Second, if preoccupied performing another task, it is easy for the expectant mother to miss instances of fetal movement. Third, fetuses tend to be most active at night, when the expectant mother is hopefully asleep, so a large portion of daily movement may go untracked.

### SUMMARY

Various embodiments are described related to a contactless fetal movement tracking device. In some embodiments, a contactless fetal movement tracking device is described. The device may comprise a housing. The device may comprise a touchscreen electronic display housed by the housing. The device may comprise a radar sensor housed by the housing, the radar sensor aimed such that when the housing is placed bedside, movement of an expectant mother within a bed is detected. The device may comprise a processing system housed by the housing, comprising one or more processors, that may receive data from the radar sensor and the touchscreen electronic display and output data to the touchscreen electronic display for presentation. The processing system may be configured to receive user input, via the touchscreen electronic display, requesting that contactless fetal movement monitoring be performed. The processing system may be configured to, in response to the user input, perform a state analysis on the expectant mother to determine that the expectant mother is present and static in the bed. The processing system may be configured to, while the state analysis indicates that the expectant mother is present and static in the bed, perform fetal movement tracking using radar data received from the radar sensor. The processing system may be configured to cause a fetal tracking report to be presented by the touchscreen electronic display based on the performed fetal movement tracking.

Embodiments of such a device may include one or more of the following features: the fetal tracking report may be indicative of fetal movement occurring during a multi-hour period of time during a previous night while the expectant mother was asleep. The performed fetal movement tracking using the radar data received from the radar sensor may be at least in part performed using a trained machine learning model. The performed fetal movement tracking using the radar data received from the radar sensor may be performed by monitoring for movement a distance from a detected vital sign of the expectant mother. The processing system may be further configured to sort instances of detected fetal movement by time. The fetal tracking report may indicate a plurality of indications of movements per unit of time. The device may further comprise a network interface that may communicate with a remote server via the Internet. Data from the fetal tracking report may be stored by the remote server in association with a user account linked to the expectant mother. The state analysis may comprise determining that the expectant mother is asleep based on the expectant mother having been present and static for at least a defined period of time. The processing system may be further configured to perform a comparison of the monitored fetal movement from a previous night with fetal movement data from an earlier time period. The fetal tracking report may indicate a result of the performed comparison. The processing system may be further configured to calculate a trend over multiple days using the monitored fetal movement, the trend indicating whether fetal movement may be generally increasing, decreasing, or staying constant over a time period comprising a plurality of nights. The fetal tracking report may indicate the calculated trend. The radar sensor may emit frequency-modulated continuous wave radar. The device may further comprise a beam steering module configured to electronically aim toward the expectant mother.

In some embodiments, a method for performing contactless fetal movement tracking is described. The method may comprise receiving user input, by a contactless fetal movement tracking device, requesting that contactless fetal movement monitoring be performed. The method may comprise based at least in part on the user input, performing, by the contactless fetal movement tracking device, a state analysis on an expectant mother to determine that the expectant mother is present and static in a bed at which the contactless fetal movement tracking device is pointed. The method may comprise performing, by the contactless fetal movement tracking device, fetal movement tracking using radar data received from a radar sensor of the contactless fetal movement tracking device while the state analysis may indicate that the expectant mother is present and static in the bed. The method may comprise presenting, by the contactless fetal movement tracking device, on an electronic display, a fetal tracking report to based on the performed fetal movement tracking.

Embodiments of such a method may include one or more of the following features: the fetal tracking report may be indicative of fetal movement occurring during a multi-hour period of time while the expectant mother was asleep. Performing fetal movement tracking using the radar data received from the radar sensor may comprise using a trained machine learning model. Performing fetal movement tracking using the radar data received from the radar sensor may comprise monitoring for movement a distance from a detected vital sign of the expectant mother. The method may further comprise sorting instances of detected fetal movement by time. The fetal tracking report may indicate a plurality of indications of movements per unit of time. The method may further comprise transmitting, via a network interface to a remote server, data included in the fetal tracking report. The method may further comprise storing, by the remote server, the data in association with a user account linked to the expectant mother. The method may further comprise calculating a trend over multiple days using the monitored fetal movement, the trend indicating whether fetal movement is generally increasing, decreasing, or staying constant over a time period comprising a plurality of nights. The fetal tracking report may indicate the calculated trend. The method may comprise receiving, via a microphone of the contactless fetal movement tracking device, a voice command requesting the fetal tracking report. The fetal tracking report may be presented in response to the received voice command.

In some embodiments, a non-transitory processor-readable medium is described. The medium may comprise processor-readable instructions configured to cause one or more processors of a contactless fetal movement tracking device to receive user input requesting that contactless fetal movement monitoring be performed. The device, based at least in part on the user input, may perform a state analysis on an expectant mother to determine that the expectant mother is present and static in a bed. The device may perform fetal movement tracking using radar data received from a radar sensor incorporated as part of the contactless fetal movement tracking device. The fetal movement tracking may be performed while the state analysis indicates that the expectant mother is present and static in the bed. The device may cause, on an electronic display of the contactless fetal movement tracking device, a fetal tracking report to based on the performed fetal movement tracking.

### BRIEF DESCRIPTION OF THE DRAWINGS

A further understanding of the nature and advantages of various embodiments may be realized by reference to the following figures. In the appended figures, similar components or features may have the same reference label. Further, various components of the same type may be distinguished by following the reference label by a dash and a second label that distinguishes among the similar components. If only the first reference label is used in the specification, the description is applicable to any one of the similar components having the same first reference label irrespective of the second reference label.

FIG. 1 illustrates a block diagram of an embodiment of a contactless fetal movement tracking system.

FIG. 2A illustrates an embodiment of a radar system that can be incorporated as part of a contactless fetal movement tracking device.

FIG. 2B illustrates an embodiment of a radar system that uses beam steering and can be incorporated as part of a contactless fetal movement tracking device.

FIG. 3 illustrates an embodiment of frequency-modulated continuous wave radar radio waves output by a radar subsystem.

FIG. 4A illustrates an embodiment of a contactless fetal movement tracking device.

FIG. 4B illustrates an exploded view of an embodiment of a contactless fetal movement tracking device.

FIG. 5 illustrates an embodiment of a state machine for determining when a person is sleeping.

FIG. 6 illustrates an embodiment of a beam-steering module for a contactless fetal movement tracking device that targets the direction in which fetal tracking is performed.

FIG. 7 illustrates an embodiment of the antenna layout of a radar subsystem that may be used in combination with a beam-steering module of a contactless fetal movement tracking device.

FIG. 8 illustrates an embodiment of a method for performing contactless fetal movement tracking.

FIG. 9 illustrates an embodiment of a user interface for indicating that a user desires to enable fetal movement tracking.

FIG. 10 illustrates an embodiment of a user interface for performing initial calibration for fetal movement tracking.

FIG. 11 illustrates an embodiment of another user interface for performing initial calibration for fetal movement tracking.

FIG. 12 illustrates an embodiment of a fetal movement tracking report presented by a contactless fetal movement tracking device.

FIG. 13 illustrates an embodiment of an expectant mother sleeping while having fetal movement tracked.

### DETAILED DESCRIPTION

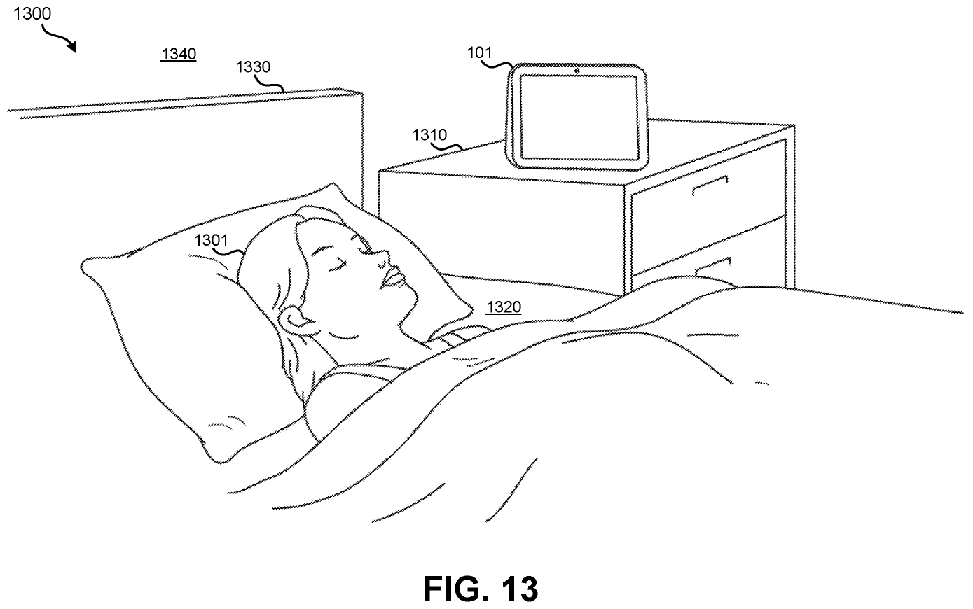

Embodiments of a contactless fetal movement tracking device and associated methods are detailed herein. A contactless fetal movement tracking device (“FMTD”) can be positioned bedside, such as shown in embodiment 1300 of FIG. 13. By being positioned bedside, such as on nightstand 1310 next to bed 1320 in which the expectant mother (“EM”) 1301 sleeps, fetal movement of a fetus can be detected and tracked throughout the night using radar without any sensor needing to be in physical contact with EM 1301 or bed 1320.

An EM may provide consent to an FMTD indicating that the EM desires to have fetal movement tracked. The EM can remain in control of data storage related to detected fetal movement. A calibration process may be performed to ensure that the FMTD is positioned and aimed correctly such that monitoring of fetal movement can occur throughout the night. Fetal movement monitoring can be performed once a state analysis is performed to determine that the EM is asleep or is at least still and lying in bed.

Radio waves, such as frequency modulated continuous wave (FMCW) radar waves, are emitted by the FMTD. In some embodiments, beam steering is electronically performed to target an abdomen of the EM and, thus, the fetus. A trained machine learning (ML) model can be used to detect fetal movement and distinguish between fetal movement and movement of the EM. Detected instances of fetal movement (e.g., kicks, rolls, etc.) can be tracked and timestamped.

A nightly fetal movement report can be prepared and presented by the FMTD that indicates information about fetal movement detected during the previous night. The nightly report can also be available on other devices, such as a computerized device that is associated with or logged in to a same user account as the FMTD. In some embodiments, the FMTD outputs information about fetal movement for multiple time periods, such as for each hour that the EM was present in bed or was determined to be asleep. For instance, the EM may be interested in the number of fetal movements per hour and, thus, the nightly report may indicate a number of detected fetal movements per hour. In some embodiments, the trained ML model can distinguish between different types of fetal movements.

The FMTD, either on its own or in combination with a remote server system, can cause information to be presented or otherwise output (e.g., via synthesized speech) that indicates longer-term trends, such as whether fetal movement is increasing, decreasing, or staying approximately the same over multiple nights (e.g., the last 1-4 weeks, the last month, etc.). An indication may be provided when the number of detected fetal movements are greater or fewer than average.

In some embodiments, the FMTD is incorporated as part of a sleep sensing device. In addition to performing the functions detailed herein related to fetal movement tracking, the FMTD may use radar to track sleeping of the EM.

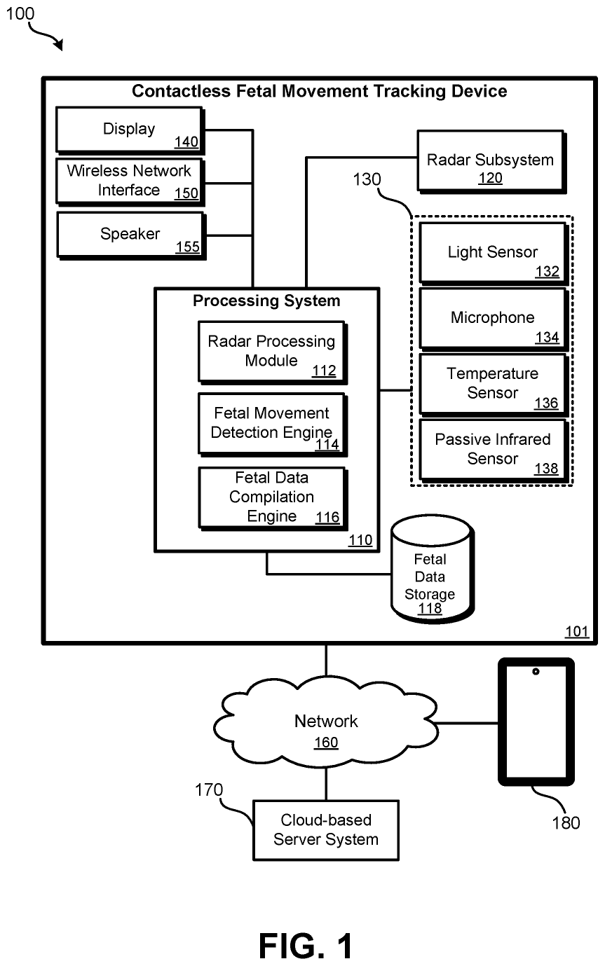

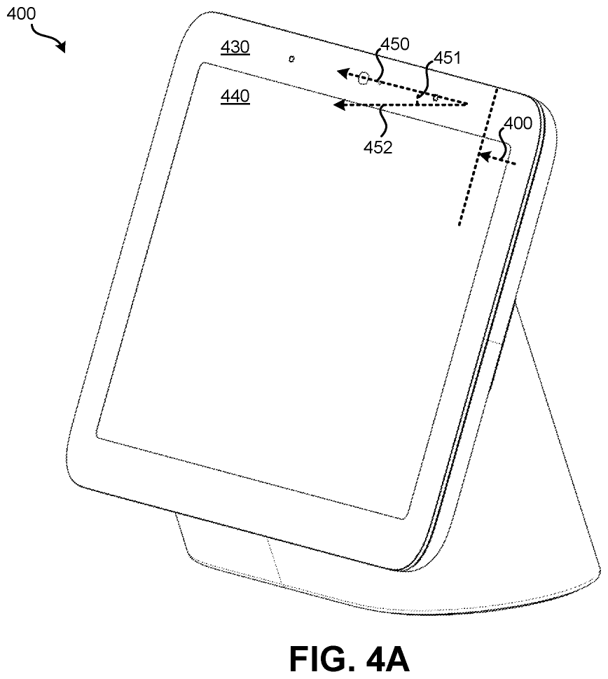

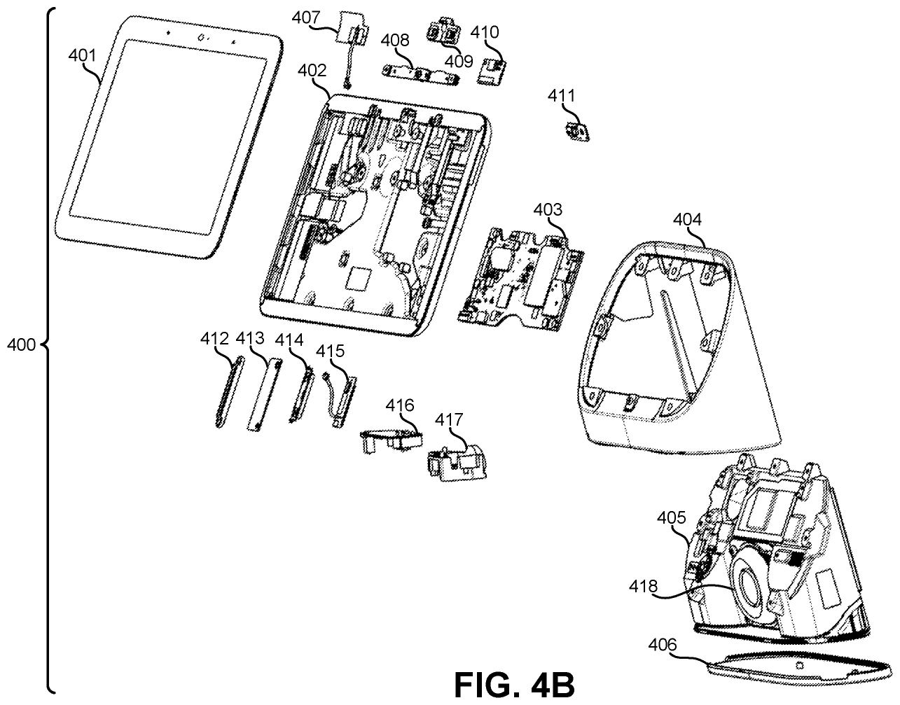

Further detail regarding such embodiments is provided in relation to the figures. FIG. 1 illustrates a block diagram of an embodiment of a fetal movement tracking system 100 (“system 100”). System 100 can include: FMTD 101; network 160; cloud-based server system 170; and computerized device 180. FMTD 101 can include: processing system 110; fetal data storage 118; radar subsystem 120; environmental sensor suite 130; display 140; wireless network interface 150; and speaker 155. Generally, FMTD 101 can include a housing that houses all of the components of FMTD 101. Further detail regarding such a housing, according to some embodiments, is provided in relation to FIG. 4A and FIG. 4B.

Processing system 110 can include one or more processors configured to perform various functions, such as the functions of: radar processing module 112; fetal movement detection engine 114, and fetal data compilation engine 116. Processing system 110 can include one or more special-purpose or general-purpose processors. Such special-purpose processors may include processors that are specifically designed to perform the functions detailed herein. Such special-purpose processors may be ASICs or FPGAs which are general-purpose components that are physically and electrically configured to perform the functions detailed herein. Such general-purpose processors may execute special-purpose software that is stored using one or more non-transitory processor-readable mediums, such as random access memory (RAM), flash memory, a hard disk drive (HDD), or a solid state drive (SSD).

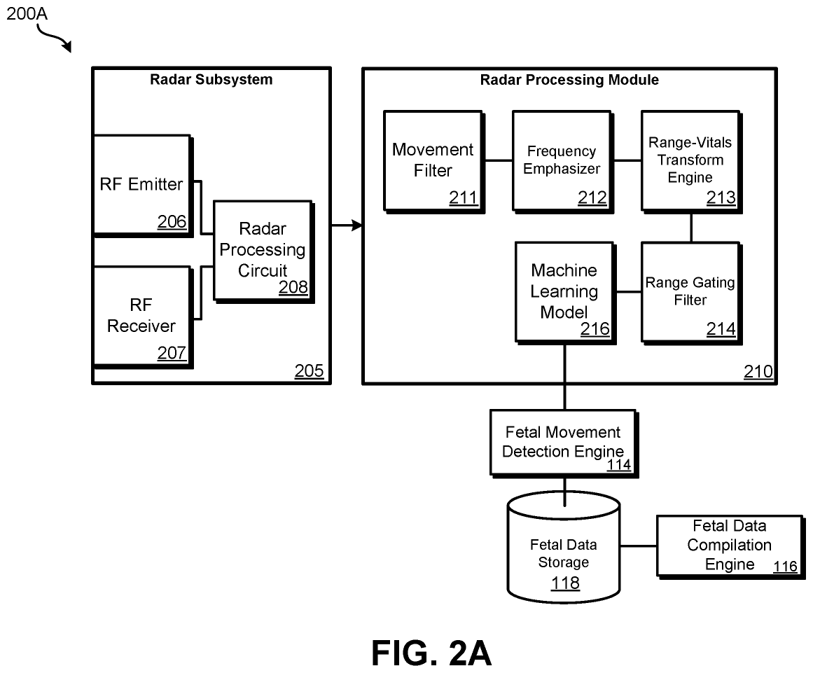

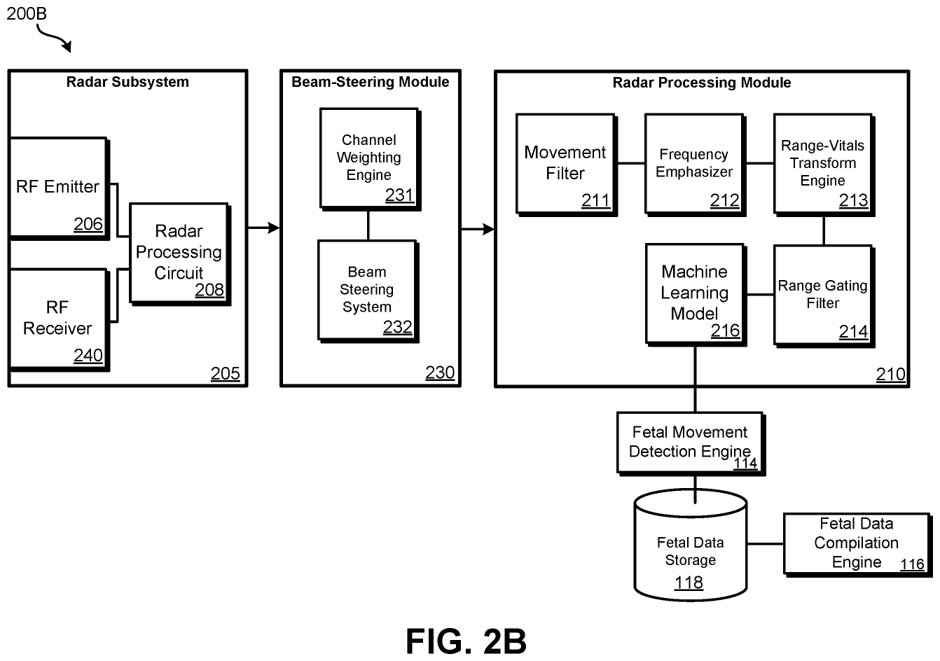

Radar subsystem 120 (also referred to as a radar sensor) can be a single integrated circuit (IC) that emits, receives, and outputs data indicative of a received, reflected waveform. The output of radar subsystem 120 may be analyzed using radar processing module 112 of processing system 110. Radar subsystem 120 does not require physical contact with an object to perform measurements. Therefore, radar subsystem 120 can be located within FMTD 101 bedside to measure fetal movement while an EM is in bed. Further detail regarding radar subsystem 120 and radar processing module 112 is provided in relation to FIG. 2.

FMTD 101 may include one or more environmental sensors, such as all, one, or some combination of the environmental sensors provided as part of environmental sensor suite 130. Environmental sensor suite 130 can include: light sensor 132; microphone 134; temperature sensor 136; and passive infrared (PIR) sensor 138. In some embodiments, multiple instances of some or all of these sensors may be present. For instance, in some embodiments, multiple microphones may be present. Light sensor 132 may be used for measuring an ambient amount of light present in the general environment of FMTD 101. Microphone 134 may be used for measuring an ambient noise level present in the general environment of FMTD 101. Temperature sensor 136 may be used for measuring an ambient temperature of the general environment of FMTD 101. PIR sensor 138 may be used to detect moving living objects (e.g., persons, pets) within the general environment of FMTD 101. Other types of environmental sensors are possible. For instance, a camera and/or humidity sensor may be incorporated as part of environmental sensor suite 130. As another example, active infrared sensors may be included. In some embodiments, some data, such as humidity data, may be obtained from a nearby weather station that has data available via the Internet. In some embodiments, active acoustic sensing methods, including, but not limited to, sonar and ultrasound, and including either single or arrayed acoustic sources and/or receivers may be implemented. Such arrangements may be used as one or more adjunct sensing modalities incorporated with the other sensors and methods described herein.

In some embodiments, one, some, or all of sensors of environmental sensor suite 130 may be external device to 101. For instance, one or more remote environmental sensors may communicate with FMTD 101, either directly (e.g., via a direct wireless communication method, via a low-power mesh network) or indirectly (e.g., through one or more other devices via the low-power mesh network, via an access point of a network, via a remote server).

FMTD 101 may include various interfaces. Display 140 can allow processing system 110 to present information for viewing by one or more users. Wireless network interface 150 can allow for communication using a wireless local area network (WLAN), such as a WiFi-based network. Speaker 155 can allow for sound, such as synthesized speech, to be output. For instance, responses to spoken commands received via microphone 134 may be output via speaker 155 and/or display 140. The spoken commands may be analyzed locally by FMTD 101 or may be transmitted via wireless network interface 150 to cloud-based server system 170 for analysis. A response, based on the analysis of the spoken command, can be sent back to FMTD 101 via wireless network interface 150 for output via speaker 155 and/or display 140. Additionally or alternatively, the speaker 155 and microphone 134 may be collectively configured for active acoustic sensing, including ultrasonic acoustic sensing. Additionally or alternatively, other forms of wireless communication may be possible, such as using a low-power wireless mesh network radio and protocol (e.g., Thread) to communicate with various smart home devices. In some embodiments, a wired network interface, such as an Ethernet connection, may be used for communication with a network. Further, the evolution of wireless communication to fifth generation (5G) and sixth generation (6G) standards and technologies provides greater throughput with lower latency which enhances mobile broadband services. 5G and 6G technologies also provide new classes of services, over control and data channels, for vehicular networking (V2X), fixed wireless broadband, and the Internet of Things (IoT). Such standards and technologies may be used for communication by FMTD 101.

The low-power wireless mesh network radio and protocol may be used for communicating with power limited devices. A power-limited device may be an exclusively battery powered device. Such devices may rely exclusively on one or more batteries for power and, therefore, the amount of power used for communications may be kept low in order to decrease the frequency at which the one or more batteries need to be replaced. In some embodiments, a power-limited device may have the ability to communicate via a relatively high power network (e.g., WiFi) and the low-power mesh network. The power-limited device may infrequently use the relatively high power network to conserve power. Examples of such power-limited devices include environmental sensors (e.g., temperature sensors, carbon monoxide sensors, smoke sensors, motion sensors, presence detectors) and other forms of remote sensors.

Notably, some embodiments of FMTD 101 do not have any still camera or video camera. By not incorporating an onboard camera, users nearby may be reassured about their privacy. For example, FMTD 101 can typically be installed in a user's bedroom. For many reasons, a user, such as an EM, would not want a camera located in such a private space or aimed toward the user while the user is sleeping. In other embodiments, FMTD 101 may have a camera, but the camera's lens may be obscured by a mechanical lens shutter. In order to use the camera, the user may be required to physically open the shutter to allow the camera to have a view of the environment of FMTD 101. The user can be assured of privacy from the camera when the shutter is closed.

Wireless network interface 150 can allow for wireless communication with network 160. Network 160 can include one or more public and/or private networks. Network 160 can include a local wired or wireless network that is private, such as a home wireless local area network. Network 160 may also include a public network, such as the Internet. Network 160 can allow for FMTD 101 to communicate with remotely located cloud-based server system 170.

Cloud-based server system 170 can provide FMTD 101 with various services. Regarding fetal movement data, cloud-based server system 170 can include processing and storage services. FMTD 101 can be linked with a particular user account. Cloud-based server system 170 can be used to store and/or analyze data collected using FMTD 101, if authorized by a user of FMTD 101. By storing data using cloud-based server system 170, a user, such as an EM, can access fetal movement data via another device, such as computerized device 180, that communicates with network 160.

Computerized device 180 can be any form of computerized device that can be used to access network 160 and access the user account linked with FMTD 101. For example, computerized device 180 can be a smartphone, tablet computer, laptop computer, desktop computer, gaming device, etc. In some embodiments, computerized device 180 can execute an installed application that is linked with the user account. For example, on a smartphone, an “app” can be installed from an “app store” that is then linked to the user account and used to access fetal movement data stored by cloud-based server system 170 and/or FMTD 101 in association with the user account.

While the embodiment of FIG. 1 involves processing system 110 performing fetal movement tracking, in other embodiments, such functions may be performed by cloud-based server system 170. Also, in addition or in alternate to fetal data storage 118 being used to store fetal data, fetal movement-related data may be stored by cloud-based server system 170, such as mapped to a common user account to which FMTD 101 is linked. If multiple users are monitored, the fetal data may be stored and mapped to a master user account or to the corresponding users' accounts.

An EM is required to provide their informed consent for fetal movement tracking. Such informed consent may involve the EM consenting to an end user agreement that involves data being used in compliance with HIPAA and/or other generally accepted security and privacy standards for health information. Periodically, the EM (and any other user) may be required to renew their consent to collection of fetal movement data, such as annually. In some embodiments, each end user may receive a periodic notification, such as via computerized device 180, that reminds each user that their fetal movement data is being collected and analyzed and offers each user the option to disable such data collection.

Cloud-based server system 170 may additionally or alternatively provide other cloud-based services. For instance, various embodiments of FMTD 101 function as a home assistant device. A home assistant device responds to vocal queries from a user. In response to detecting a vocal trigger phrase being spoken, FMTD 101 can record and temporarily store the recorded audio. A stream of the recorded audio may be transmitted to cloud-based server system 170 for analysis or processed locally. Cloud-based server system 170 may perform a speech recognition process, use a natural language processing engine to understand the query from the user, and provide a response to be output by FMTD 101 as synthesized speech, an output to be presented on display 140, and/or a command to be executed by FMTD 101 (e.g., raise the audio volume of FMTD 101, present fetal movement data from the past night) or sent to some other smart home device. Further, queries or commands may be submitted to cloud-based server system 170 via display 140, which may be a touchscreen display that also functions as a user input device. For instance, FMTD 101 may be used to control various smart home devices or home automation devices. Such commands may be sent directly by FMTD 101 to the device to be controlled or may be sent via cloud-based server system 170.

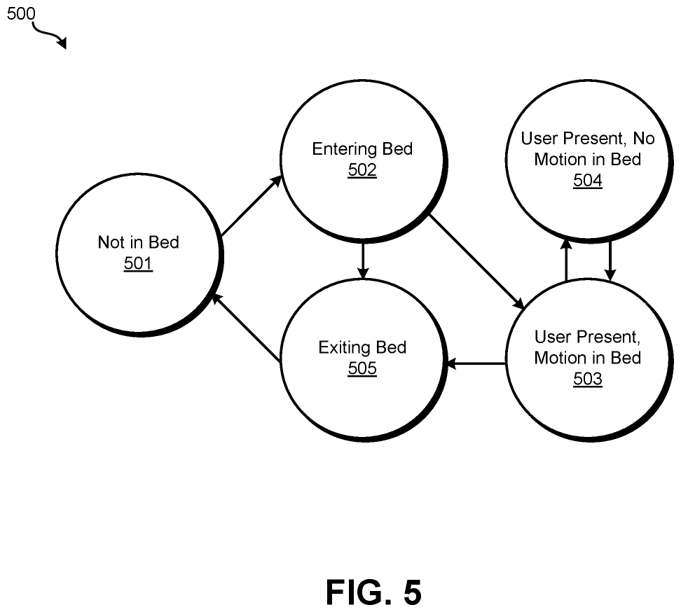

Based on data output by radar processing module 112, fetal movement detection engine 114 may be used to monitor fetal movement. Fetal movement detection engine 114 may use a state machine, such as detailed in relation to FIG. 5, to determine whether the EM is likely awake or asleep. For example, if an EM is determined to be in bed and still for at least a defined period of time, the EM may be identified as asleep. Further detail regarding fetal movement detection engine 114 and fetal data compilation engine 116 is provided in relation to FIG. 2A.

Notably, some embodiments of FMTD 101 also function as a sleep tracking device. Details of how FMTD 101 can additionally function as a sleep tracking device are provided in U.S. patent application Ser. No. 16/990,746, U.S. patent application Ser. No. 16/990,705, U.S. patent application Ser. No. 16/990,714, U.S. patent application Ser. No. 16/990,720, and U.S. patent application Ser. No. 16/990,726, each of which is hereby incorporated by reference in their entirety for all purposes.

FIG. 2A illustrates an embodiment of a fetal movement tracking system 200A (“system 200A”). System 200A can include radar subsystem 205 (which can represent an embodiment of radar subsystem 120); and radar processing module 210 (which can represent an embodiment of radar processing module 112).

Radar subsystem 205 may include RF emitter 206, RF receiver 207, and radar processing circuit 208. RF emitter 206 can emit radio waves, such as in the form of continuous-wave (CW) radar. RF emitter 206 may use frequency-modulated continuous-wave (FMCW) radar. The FMCW radar may operate in a burst mode or continuous sparse-sampling mode. In burst mode, a frame or burst of multiple chirps, with the chirps spaced by a relatively short period of time, may be output by RF emitter 206. Each frame may be followed by a relatively long amount of time until a subsequent frame. In a continuous sparse-sampling mode, frames or bursts of chirps are not output; rather chirps are output periodically. The spacing of chirps in the continuous sparse sampling mode may be greater in duration than the spacing between chirps within a frame of the burst mode. In some embodiments, radar subsystem 205 may operate in a burst mode, but output raw chirp waterfall data for each burst may be combined (e.g., averaged) together to create simulated continuous sparse-sampled chirp waterfall data. In some embodiments, raw waterfall data gathered in burst mode may be preferable for gesture detection while raw waterfall data gathered in a continuously sparse sampling mode may be preferable for sleep tracking, vital sign detection, and, generally, health monitoring. Gesture detection may be performed by other hardware or software components that use the output of radar subsystem 205 that are not illustrated.

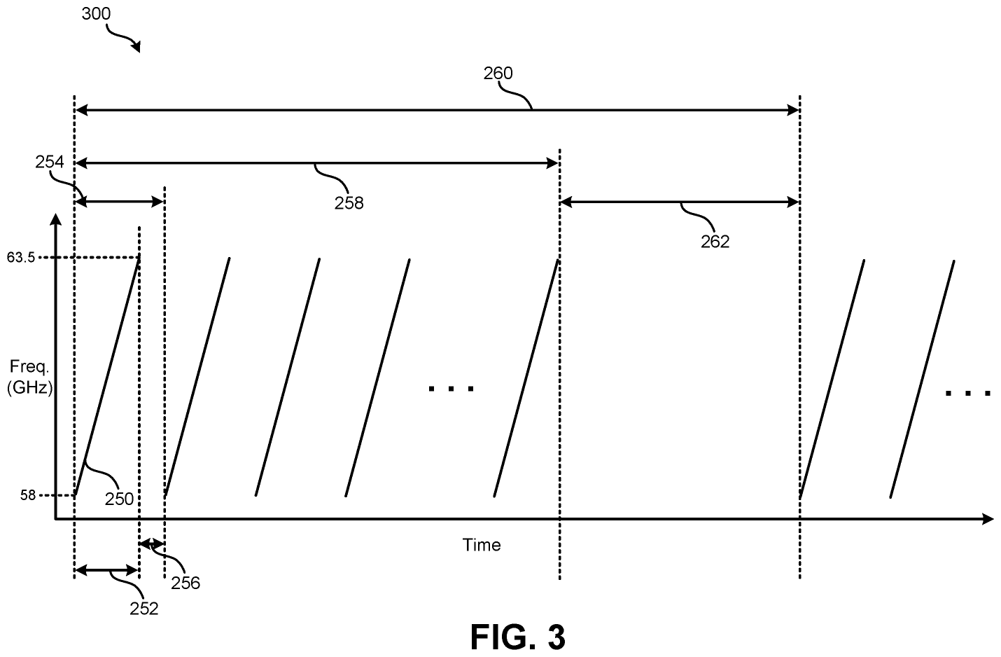

RF emitter 206 may include one or more antennas and may transmit at or about 60 GHz. The frequency of radio waves transmitted may repeatedly sweep from a low to high frequency (or the reverse). The power level used for transmission may be very low such that radar subsystem 205 has an effective range of several meters or an even shorter distance. Further detail regarding the radio waves generated and emitted by radar subsystem 205 is provided in relation to FIG. 3.

RF receiver 207 includes one or more antennas, distinct from the transmit antenna(s), and may receive radio wave reflections off of nearby objects of radio waves emitted by RF emitter 206. The reflected radio waves may be interpreted by radar processing circuit 208 by mixing the radio waves being transmitted with the reflected received radio waves, thereby producing a mixed signal that can be analyzed for distance. Based on this mixed signal, radar processing circuit 208 may output raw waveform data, which can also be referred to as the raw chirp waterfall data for analysis by a separate processing entity. Radar subsystem 205 may be implemented as a single integrated circuit (IC) or radar processing circuit 208 may be a separate component from RF emitter 206 and RF receiver 207. In some embodiments, radar subsystem 205 is integrated as part of FMTD 101 such that RF emitter 206 and RF receiver 207 are pointing in a same direction as display 140. In other embodiments, an external device that includes radar subsystem 205 may be connected with FMTD 101 via wired or wireless communication. For example, radar subsystem 205 may be an add-on device to a home assistant device.

For radar subsystem 205, if FMCW is used, an unambiguous FMCW range can be defined. Within this range, a distance to objects can be accurately determined. However, outside of this range, a detected object could be incorrectly interpreted as nearer than an object within the unambiguous range. This incorrect interpretation can be due to the frequency of the mixed signal and the sampling rate of the ADC used by the radar subsystem to convert the received analog signals to digital signals. If the frequency of the mixed signal is above the Nyquist rate of the sampling of the ADC, the digital data output by the ADC representative of the reflected radar signal can be incorrectly represented (e.g., as a lower frequency indicative of a closer object).

When using FMTD 101 to monitor fetal movement, an EM user may be instructed that the user should be the closest person to the device 201. However, it may be possible that another person or an animal is present within the bed. It may be necessary to define the unambiguous FMCW range to be far enough, such as two meters, such that both persons (or, approximately the width of the bed) fall within the unambiguous FMCW range of radar subsystem 205. Two meters may be an ideal distance since this distance is approximately the width of a large commercially available bed (e.g., a king size bed).

Raw waveform data may be passed from radar subsystem 205 to radar processing module 210. The raw waveform data passed to radar processing module 210 may include waveform data indicative of continuous sparse reflected chirps due to radar subsystem 205 operating in a continuous sparse sampling mode or due to radar subsystem 205 operating in a burst mode and a conversion process to simulate raw waveform data produced by radar subsystem 205 operating in a continuous sparse sampling mode being performed. Processing may be performed to convert burst sampled waveform data to continuous sparse samples using an averaging process, such as each reflected group of burst radio waves being represented by a single averaged sample. Radar processing module 210 may include one or more processors. Radar processing module 210 may include one or more special-purpose or general-purpose processors. Special-purpose processors may include processors that are specifically designed to perform the functions detailed herein. Such special-purpose processors may be ASICs or FPGAs which are general-purpose components that are physically and electrically configured to perform the functions detailed herein. General-purpose processors may execute special-purpose software that is stored using one or more non-transitory processor-readable mediums, such as random access memory (RAM), flash memory, a hard disk drive (HDD), or a solid state drive (SSD). Radar processing module 210 may include: movement filter 211; frequency emphasizer 212; range-vitals transform engine 213; range gating filter 214; spectral summation engine 215; and machine learning model 216. Each of the components of radar processing module 210 may be implemented using software, using firmware, or as specialized hardware.

The raw waveform data output by radar subsystem 205 may be received by radar processing module 210 and first processed using movement filter 211. In some embodiments, it is important that movement filter 211 is the initial component used to perform filtering. That is, the processing performed by radar processing module 210 is not commutative in some embodiments. Typically, vital sign determination, fetal monitoring, and sleep monitoring may occur when a monitored EM user is sleeping or attempting to sleep in a bed. In such an environment, there may typically be little movement. Such movement may be attributed to the EM user moving within the bed (e.g., rolling over while trying to get to sleep or while asleep), the EM vital signs, including movement due to breathing and movement due to the monitored user's heartbeat, and the fetus's movement. In such an environment, a large portion of emitted radio waves from RF emitter 206 may be reflected by static objects in the vicinity of the monitored user, such as a mattress, box spring, bed frame, walls, furniture, bedding, etc. Therefore, a large portion of the raw waveform data received from radar subsystem 205 may be unrelated to user movements, the user's vital measurements, and the fetus's movement (and, possibly vital measurements).

Movement filter 211 may include a waveform buffer that buffers “chirps” or slices of received raw waveform data. For instance, sampling may occur at a rate of 10 Hz. In other embodiments, sampling may be slower or faster. Movement filter 211 may buffer twenty seconds of received raw waveform chirps in certain embodiments. In other embodiments, a shorter or longer duration of buffered raw waveform data is buffered. This buffered raw waveform data can be filtered to remove raw waveform data indicative of stationary objects. That is, for objects that are moving, such as a monitored user's chest, the user's heartbeat and breathing rate will affect the distance and velocity measurements made by radar subsystem 205 and output to movement filter 211. This movement of the user will result in “jitter” in the received raw waveform data over the buffered time period. More specifically, jitter refers to the phase shifts caused by moving objects reflecting emitted radio waves. Rather than using the reflected FMCW radio waves to determine a velocity of the moving objects, the phase shift induced by the motion in the reflected radio waves can be used to measure vital statistics, including heartrate and breathing rate, as detailed herein.

For stationary objects, such as furniture, a zero phase shift (i.e., no jitter) will be present in the raw waveform data over the buffered time period. Movement filter 211 can subtract out such raw waveform data corresponding to stationary objects such that motion-indicative raw waveform data is passed to frequency emphasizer 212 for further analysis. Raw waveform data corresponding to stationary objects may be discarded or otherwise ignored for the remainder of processing by radar processing module 210.

If radar processing module 210 is additionally being used to monitor vital signs, frequency emphasizer 212 may work in conjunction with range-vitals transform engine 213 to determine the one (e.g., breathing) or two (e.g., breathing plus heartbeat) frequency components of the raw waveform data. Frequency emphasizer 212 may use frequency windowing, such as a 2D Hamming window (other forms of windowing are possible, such as a Hann window), to emphasize important frequency components of the raw waveform data and to deemphasize or remove waveform data that is attributable to spectral leakage outside of the defined frequency window. Such frequency windowing may decrease the magnitude of raw waveform data that is likely due to processing artifacts. The use of frequency windowing can help reduce the effects of data-dependent processing artifacts while preserving data relevant for being able to separately determine heartrate and breathing rate. If vital sign monitoring is not being performed, frequency emphasizer 212 may not be present or used.

If radar processing module 210 is additionally being used to monitor vital signs, range-vitals transform engine 213 analyzes the received motion-filtered waveform data to identify and quantify the magnitude of movement at specific frequencies. More particularly, range-vitals transform engine 213 analyzes phase jitter over time to detect relatively small movements due to a user's vital signs that have a relatively low frequency, such as breathing rate and heart rate. The analysis of range-vitals transform engine 213 may assume that the frequency components of the motion waveform data are sinusoidal. Further, the transform used by range-vitals transform engine 213 can also identify the distance at which the frequency is observed. Frequency, magnitude, and distance can all be determined at least in part because radar subsystem 205 uses an FMCW radar system. Again here, if vital sign monitoring is not being performed, frequency emphasized 212 may not be present or used.

Range-vitals transform engine 213 can perform a series of Fourier transform (FT) to determine the frequency components of the received raw waveform data output by frequency emphasizer 212. Specifically, a series of fast Fourier transform (FFT) may be performed by range-vitals transform engine 213 to determine the specific frequencies and magnitudes of waveform data at such frequencies.

Waveform data obtained over a period of time can be expressed in multiple dimensions. A first dimension (e.g., along the y-axis) can relate to multiple samples of waveform data from a particular chirp and a second dimension (e.g., along the x-axis) relates to a particular sample index of waveform data gathered across multiple chirps. A third dimension of data (e.g., along the z-axis) is present indicative of the intensity of the waveform data.

Multiple FFTs may be performed based on the first and second dimension of the waveform data. FFTs may be performed along each of the first and second dimensions: an FFT may be performed for each chirp and an FFT may be performed for each particular sample index across multiple chirps that occurred during the period of time. An FFT performed on waveform data for a particular reflected chirp can indicate one or more frequencies, which, in FMCW radar, are indicative of the distances at which objects are present that reflected emitted radio waves. An FFT performed for a particular sample index across multiple chirps can measure the frequency of phase jitter across the multiple chirps. Therefore, the FFT of the first dimension can provide the distance at which movement (e.g., fetal movement) is present and the FFT of the second dimension can provide frequency components of the movement. The output of the FFTs performed across the two dimensions is indicative of: 1) the frequencies of movement; 2) the ranges at which the movement were measured; and 3) the magnitudes of the measured frequencies. In particular, for detecting fetal movement, the distance from radar subsystem 205 to the movement can be important to distinguish from movement of the EM (e.g., a foot twitch).

Range gating filter 214 is used to monitor a defined range of interest and exclude waveform data due to movement beyond the defined range of interest. For arrangements detailed herein, the defined range of interest may be 0 to 1 meter. In some embodiments, this defined range of interest may be different or possibly set by a user (e.g., via a training or setup process) or by a service provider. In some embodiments, a goal of this arrangement may be to monitor the one person closest to the device (and exclude or segregate data for any other person farther away, such as a person sleeping next to the person being monitored). Therefore, range-vitals transform engine 213 and range gating filter 214 serve to segregate, exclude, or remove movement data attributed to objects outside of the defined range of interest and sum the energy of movement data attributed to objects within the defined range of interest. The output of range gating filter 214 may include data that has a determined range within the permissible range of range gating filter 214. The data may further have a frequency dimension and a magnitude. Therefore, the data may possess three dimensions.

Machine learning model 216 can receive processed radar data from range gating filter 214 (or directly from an earlier component, such as movement filter 211) to perform a machine learning-based analysis on the data. The input to machine learning model 216 may include data representative of: 1) the frequencies of movement; 2) the ranges at which the movement was detected; and 3) the magnitude of the measured frequencies. From this data, machine learning model 216 may have been trained to output a confidence that motion detected using radar subsystem 205 is due to fetal movement. A high confidence that fetal movement is present may only be determined when the EM is determined to be present and still.

In some embodiments, one or more additional machine learning models may be present. Another machine learning model may use data from range gating filter 214 to determine if the EM is present and still (and, possibly, asleep). For example, based on vital signs (e.g., breathing, heartbeat) being detected with little or no other movement, it can be determined that the EM is present and is still. If the EM has been still for at least a predefined period of time, an assumption can be made that the EM is asleep.

As one possible form of an ML model that can be used for the one or more machine learning models, trained neural networks may be used. A trained neural network ML model may be initially trained using a large set of input training data of amplitude, frequency, and range data that has been properly tagged with a classification as either corresponding to a fetal movement or not (or, in some embodiments, the specific type of fetal movement). For example, the neural network may analyze the spectral sparsity of the movement (fetal movement may be a significantly spread spectrum rather than being concentrated at particular frequencies due to kicks and rolls not being sinusoidal in nature). The neural network may be a fully connected neural network that is not time-dependent. In some embodiments, a machine-learning arrangement, classifier, or form of artificial intelligence other than a neural network may be used.

A similar strategy can be used for training an NN ML model for determining sleep state. An expert may monitor many instances of subjects going to bed, sleeping, and waking. The expert's classifications can then be used to truth-tag amplitude, frequency, and range data from radar processing module 210. This truth-tagged data can then be used to train a model for sleep state detection, such as in accordance with the states indicated in FIG. 5.

The output of machine learning model 216 (and possibly the output of one or more other machine learning models, such as an ML model that detects sleep state) can be received by fetal movement detection engine 114. In some embodiments, fetal movement detection engine 114 relies only on the confidence values of fetal movement output by machine learning model 216. Fetal movement detection engine 114 may log an instance of fetal movement when the machine learning model 216 outputs a confidence above a threshold. Fetal movement detection engine 114 may determine that at least a minimum amount of time has elapsed since a previous logged instance of fetal movement.

In some embodiments fetal movement detection engine 114 takes input from multiple sources. For instance, in addition to receiving data from machine learning model 216, the fetal movement detection engine may receive data from an ML model that determines whether the EM is sleeping. For fetal movement to be logged, it may be required that the EM be determined to be asleep. Such an arrangement can help prevent small movements of the EM from being logged as fetal movement.

Fetal data storage 118 may be used to store time-stamped indications of fetal movement. In some embodiments, this data is only stored locally at FMTD 101. Fetal data compilation engine 116 may use the data from fetal data storage 118 to create a fetal movement report for the EM. The fetal movement report may be automatically presented in the morning or upon request. Information included in the report that is compiled by fetal data compilation engine 116 from fetal data storage 118 can include: total number of fetal movements during the night; fetal movements by some time period within the night (e.g., per hour); average number of fetal movements per hour. This fetal information may be presented in combination with sleep information about the EM, if collected.

Fetal data compilation engine 116, in addition to compiling information about the previous night, may determine various trends about fetal movement and the EM over a longer period of time (e.g., week, multiple weeks, month, multiple months, etc.). Using data from fetal data storage 118, fetal data compilation engine 116 may include information in the report that indicates whether fetal movement is generally: increasing over time, decreasing over time, or staying about the same. Any noted decreases may be cause for the EM to have the fetus checked by a doctor.

The functions of fetal movement detection engine 114, fetal data storage 118, and fetal data compilation engine 116 may be performed using one or more processors of FMTD 101. Alternatively, data may be transmitted to cloud-based server system 170 for analysis and storage. Therefore, the functions of such components can be integrated as part of cloud-base server system 170.

Further, in some embodiments, radar processing module 210 may be wholly or partly located remotely from FMTD 101. While radar subsystem 205 may need to be local to the monitored user, the processing of radar processing module 210 may be moved to cloud-based server system 170. In other embodiments, a smart home device that is in local communication (e.g., via a LAN or WLAN) with FMTD 101 may perform some or all of the processing of radar processing module 210. In some embodiments, a local communication protocol, such as involving a mesh network, can be used to transmit the raw waveform data to the local device that will be performing the processing. Such communication protocols can include Wi-Fi, Bluetooth, Thread, or communication protocols of the IEEE 802.11 and 802.15.4 families. Similar to the processing, storage of the sleep data and vital statistic data may occur at cloud-based server system 170 or another smart home device in the home at which FMTD 101 is located. In still other embodiments, radar processing module 210 may be incorporated with radar subsystem 205 as a single component or system of components.

FIG. 2B illustrates an embodiment of a fetal movement tracking system 200B (“system 200B”) which can perform beam targeting. Beam targeting performed by using beam-steering module 230 can focus on radar reflections from a region in which a user may be present and ignore or at least decrease the use of radar reflections from objects that cause interference, such as a nearby wall or large object.

Radar subsystem 240 may contain multiple antennas to receive reflected radar radio waves. In some embodiments, three antennas may be present. These antennas may be aligned in an “L” pattern, such that two antennas are horizontally orthogonal and two antennas are vertically orthogonal with one of the antennas being used in both the horizontal arrangement and vertical arrangement. By analyzing the phase difference in received radar signals, a weighting may be applied to target the received radar beam vertically and/or horizontally. In other embodiments, the antennas may be aligned in a different pattern and/or the beam targeting may be performed using a single receive antenna and multiple transmit antennas or by both multiple transmit and multiple receive antennas.

Vertical targeting may be performed to compensate for a vertical tilt of the device in which system 200B is incorporated. For instance, as discussed below in relation to FIG. 4A, the face of FMTD 400 may be tilted with respect to where a user will typically be sleeping.

Horizontal targeting may be performed to compensate for emitted radar being pointed towards an object that causes interference. For instance, if a user's bed headboard is against a wall, the headboard and/or wall may occupy a significant portion of the field-of-view of radar subsystem 120. (Such an example of a headboard is shown in FIG. 13 as headboard 1330 and wall 1340.) Radar reflections from the headboard and/or wall are not useful in determining data about the user; therefore, it may be beneficial to deemphasize reflections from the wall and/or headboard and emphasize reflections obtained away from the wall and/or headboard. Therefore, the receive beam may be steered horizontally away from the wall and the headboard by weighting applied to the received radar signals.

In system 200B, beam-steering module 230 is present to perform processing on the raw chirp waterfall received from radar subsystem 205 before processing is performed by radar processing module 210. Therefore, beam-steering module 230 can function as a preprocessing module prior to the analysis of radar processing module 210 and can serve to emphasize regions where an EM is expected to be present. Beam-steering module 230 may be implemented using hardware, software, or firmware; therefore, beam-steering module 230 may be implemented using the same one or more processors as radar processing module 210.

Beam-steering module 230 can include channel weighting engine 231 and beam steering system 232. Channel weighting engine 231 can be used to perform a training process to determine a series of weightings to be applied to received radar signals from each antenna prior to the received radar signals being mixed together. Channel weighting engine 231 may perform a training process when a monitored region is determined to be empty. During such time, the strength of signals received from large static objects (e.g., walls, headboards) can be analyzed and weightings can be set to steer the beam horizontally (and possibly vertically) away from such objects. Therefore, the amount of reflection in a static environment may be minimized for a particular distance range (e.g., up to one meter) from the device by channel weighting engine 231 steering the receive radar beam. Such training may also be performed when a user is present. That is, the receive beam of radar subsystem 205 can be steered to where motion is detected, or to where vital signs of an EM are present.

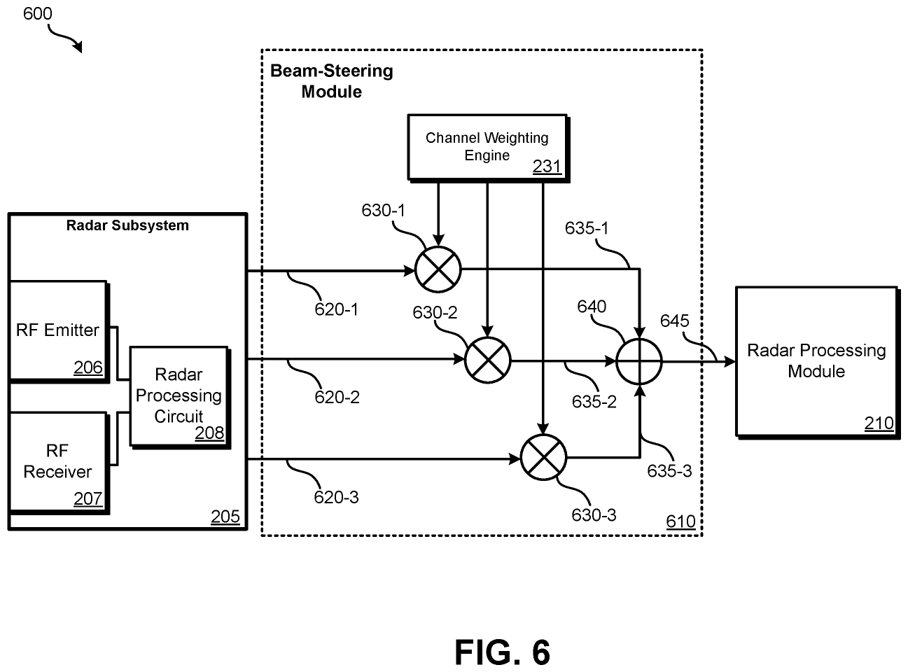

The weightings determined by channel weighting engine 231 may be used by beam steering system 232 to individually apply a weight to the received reflected radar signals of each antenna. The received signals from each antenna may be weighted, then mixed together for processing by radar processing module 210. Further detail regarding how various embodiments of beam-steering module 230 may be implemented are detailed in relation to FIG. 6. Beam-steering module 230 can be used in combination with any other embodiment detailed herein.

FIG. 3 illustrates an embodiment of chirp timing diagram 200C for frequency modulated continuous wave (FMCW) radar radio waves output by a radar subsystem. Chirp timing diagram 200C is not to scale. Radar subsystem 205 may generally output radar in the pattern of chirp timing diagram 200C. Chirp 250 represents a continuous pulse of radio waves that sweeps up in frequency from a low frequency to a high frequency. In other embodiments, individual chirps may continuously sweep down from a high frequency to a low frequency, from a low frequency to a high frequency, and back to a low frequency, or from a high frequency to a low frequency and back to a high frequency. In some embodiments, the low frequency is 58 GHz and the high frequency is 63.5 GHZ. (For such frequencies, the radio waves may be referred to as millimeter waves.) In some embodiments, the frequencies are between 57 and 64 GHz. The low frequency and the high frequency may be varied by embodiment. For instance, the low frequency and the high frequency may be between 45 GHz and 80 GHz. The frequencies may be selected at least in part to comply with governmental regulation. In some embodiments, each chirp includes a linear sweep from a low frequency to a high frequency (or the reverse). In other embodiments, an exponential or some other pattern may be used to sweep the frequency from low to high or high to low.

Chirp 250, which can be representative of all chirps in chirp timing diagram 300, may have chirp duration 252 of 128 μs. In other embodiments, chirp duration 252 may be longer or shorter, such as between 50 μs and 1 ms. In some embodiments, a period of time may elapse before a subsequent chirp is emitted. Inter-chirp pause 256 may be 205.33 μs. In other embodiments, inter-chirp pause 256 may be longer or shorter, such as between 10 μs and 1 ms. In the illustrated embodiment, chirp period 254, which includes chirp 250 and inter-chirp pause 256, may be 333.33 μs. This duration varies based on the selected chirp duration 252 and inter-chirp pause 256.

A number of chirps that are output, separated by inter-chirp pauses, may be referred to as frame 258 or frame 258. Frame 258 may include twenty chirps. In other embodiments, the number of chirps in frame 258 may be greater or fewer, such as between 1 and 100. The number of chirps present within frame 258 may be determined based upon a maximum amount of power that is desired to be output within a given period of time. The FCC or other regulatory agency may set a maximum amount of power that is permissible to be radiated into an environment. For example, a duty cycle requirement may be present that limits the duty cycle to less than 10% for any 33 ms time period. In one particular example in which there are twenty chirps per frame, each chirp can have a duration of 128 μs, each frame being 33.33 ms in duration. The corresponding duty cycle is (20 frames)*(0.128 ms)/(33.33 ms), which is about 7.8%. By limiting the number of chirps within frame 258 prior to an inter-frame pause, the total amount of power output may be limited. In some embodiments, the peak EIRP (effective isotropically radiated power) may be 13 dBm (20 mW) or less, such as 12.86 dBm (19.05 mW). In other embodiments, the peak EIRP is 15 dBm or less and the duty cycle is 15% or less. In some embodiments, the peak EIRP is 20 dBm or less. That is, at any given time, the amount of power radiated by the radar subsystem might never exceed such values. Further, the total power radiated over a period of time may be limited.

Frames may be transmitted at a frequency of 30 Hz (33.33 ms) as shown by time period 260. In other embodiments, the frequency may be higher or lower. The frame frequency may be dependent on the number of chirps within a frame and the duration of inter-frame pause 262. For instance, the frequency may be between 1 Hz and 50 Hz. In some embodiments, chirps may be transmitted continuously, such that the radar subsystem outputs a continuous stream of chirps interspersed with inter-chirp pauses. Tradeoffs can be made to save on the average power consumed by the device due to transmitting chirps and processing received reflections of chirps. Inter-frame pause 262 represents a period of time when no chirps are output. In some embodiments, inter-frame pause 262 is significantly longer than the duration of frame 258. For example, frame 258 may be 6.66 ms in duration (with chirp period 254 being 333.33 μs and 20 chirps per frame). If 33.33 ms occur between frames, inter-frame pause 262 may be 26.66 ms. In other embodiments, the duration of inter-frame pause 262 may be larger or smaller, such as between 15 ms and 40 ms.

In the illustrated embodiment of FIG. 2C, a single frame 258 and the start of a subsequent frame are illustrated. It should be understood that each subsequent frame can be structured similarly to frame 258. Further, the transmission mode of the radar subsystem may be fixed. That is, regardless of whether a user (or, specifically, an EM) is present or not, the time of day, or other factors, chirps may be transmitted according to chirp timing diagram 200C. Therefore, in some embodiments, the radar subsystem always operates in a single transmission mode, regardless of the state of the environment or the activity attempting to be monitored. A continuous train of frames similar to frame 258 may be transmitted while FMTD 101 is powered on.

FIG. 4A illustrates an embodiment of a contactless fetal movement tracking device 400 (“device 400”). Device 400 may have a front surface that includes a front transparent screen 440 such that a display is visible. Such a display may be a touchscreen. Surrounding front transparent screen 440 may be an optically opaque region, referred to as bezel 430, through which radar subsystem 205 may have a field-of-view of the environment in front of device 400.

For purposes of the immediately following description, the terms vertical and horizontal describe directions relative to the bedroom in general, with vertical referring to a direction perpendicular to the floor and horizontal referring to a direction parallel to the floor. Since the radar subsystem, which may be an Infineon® BGT60 radar chip, is roughly planar and is installed generally parallel to bezel 430 for spatial compactness of the device as a whole, and since the antennas within the radar chip lie in the plane of the chip, then, without beam targeting, a receive beam of radar subsystem 120 may be pointed in direction 450 that is generally normal to bezel 430. Due to a departure tilt of bezel 430 away from a purely vertical direction, which is provided in some embodiments to be about 25 degrees in order to facilitate easy user interaction with a touchscreen functionality of the transparent screen 440, direction 450 may point upwards from horizontal by departure angle 451. Assuming device 400 will typically be installed on a bedside platform (e.g., nightstand) that is roughly the same height as the top of a mattress on which a user, such as an EM, will sleep, it may be beneficial for the receive beam of radar subsystem 120 to be targeted in horizontal direction 452 or an approximately horizontal (e.g., between −5° and 5° from horizontal) direction. Therefore, vertical beam targeting can be used to compensate for departure angle 451 of the portion of device 400 in which radar subsystem 120 is present.

FIG. 4B illustrates an exploded view of an embodiment of contactless fetal movement tracking device 400. Device 400 can include: display assembly 401; display housing 402; main circuit board 403; neck assembly 404; speaker assembly 405; base plate 406; mesh network communication interface 407; top daughterboard 408; button assembly 409; radar assembly 410; microphone assembly 411; rocker switch bracket 412; rocker switch board 413; rocker switch button 414; Wi-Fi assembly 415; power board 416; and power bracket assembly 417. Device 400 can represent an embodiment of how FMTD 101 may be implemented.

Display assembly 401, display housing 402, neck assembly 404, and base plate 406 may collectively form a housing that houses all of the remaining components of device 400. Display assembly 401 may include an electronic display, which can be a touchscreen, that presents information to a user. Display assembly 401 may, therefore, include a display screen, which can include a metallic plate of the display that can serve as a grounding plane. Display assembly 401 may include transparent portions away from the metallic plate that allow various sensors a field of view in the general direction in which display assembly 401 is facing. Display assembly 401 may include an outer surface made of glass or transparent plastic that serves as part of the housing of device 400.

Display housing 402 may be a plastic or other rigid or semi-rigid material that serves as a housing for display assembly 401. Various components, such as main circuit board 403; mesh network communication interface 407; top daughterboard 408; button assembly 409; radar assembly 410; and microphone assembly 411 may be mounted on display housing 402. Mesh network communication interface 407; top daughterboard 408; radar assembly 410; and microphone assembly 411 may be connected to main circuit board 403, using flat wire assemblies. Display housing may be attached with display assembly 401, using an adhesive.

Mesh network communication interface 407 may include one or more antennas and may enable communication with a mesh network, such as a Thread-based mesh network. Wi-Fi assembly 415 may be located a distance from mesh network communication interface 407 to decrease the possibility of interference. Wi-Fi assembly 415 may enable communication with a Wi-Fi based network.

Radar assembly 410, which can include radar subsystem 120 or radar subsystem 205, may be positioned such that its RF emitter and RF receiver are away from the metallic plate of display assembly 401 and are located a significant distance from mesh network communication interface 407 and Wi-Fi assembly 415. These three components may be arranged in approximately a triangle to increase the distance between the components and decrease interference. For instance, in device 400, a distance of at least 74 mm between Wi-Fi assembly 415 and radar assembly 410 may be maintained. A distance of at least 98 mm between mesh network communication interface 407 and radar assembly 410 may be maintained. Additionally, distance between radar assembly 410 and speaker 418 may be desired to minimize the effect of vibrations on radar assembly 410 that may be generated by speaker 418. For instance, for device 400, a distance of at least 79 mm between radar assembly 410 and speaker 418 may be maintained. Additionally, distance between the microphones and radar assembly 410 may be desired to minimize any possible interference from the microphones on received radar signals. Top daughterboard 408 may include multiple microphones. For instance, at least 12 mm may be maintained between a closest microphone of top daughterboard 408 and radar assembly 410.

Other components may also be present. A third microphone assembly may be present, microphone assembly 411, which may be rear-facing. Microphone assembly 411 may function in concert with the microphones of top daughterboard 408 to isolate spoken commands from background noise. Power board 416 may convert power received from an AC power source to DC to power the components of device 400. Power board 416 may be mounted within device 400 using power bracket assembly 417. Rocker switch bracket 412, rocker switch board 413, and rocker switch button 414 may be collectively used to receive user input, such as up/down input. Such input may be used, for example, to adjust a volume of sound output through speaker 418. As another user input, button assembly 409 may include a toggle button that a user can actuate. Such a user input may be used to activate and deactivate all microphones, such as for when the user desires privacy and/or does not want device 400 to respond to voice commands.

As previously detailed, a machine learning model may be used to determine if the EM is asleep or is at least motionless (except for vital signs) in bed. Being motionless may be a precondition for detection of fetal movement. FIG. 5 illustrates an embodiment of a state machine 500 for determining when the EM is sleeping. Based upon data output by radar processing module 112, a sleep state detection engine may determine whether the EM is sleeping using state machine 500. It should be understood that, in some embodiments, fetal movement detection engine 114 is incorporated as part of the functionality of radar processing module 112 and does not exist as a separate module. State machine 500 may include five possible sleep states: entering bed state 501; not in bed state 502; motion in bed state 503; no motion in bed state 505; and exiting bed state 504.

If no motion-indicative waveform data is present, this may be indicative that the EM is not in bed. An EM who is in bed can be expected to always be moving in at least small amounts due to their vital signs. Therefore, if zero movement is observed, the EM may be judged to be in state 501. Following state 501 being determined; the next possible state that may be determined is state 502. In state 502, the monitored EM is entering bed. Significant EM motion across the frequency spectrum may be sensed due to the non-sinusoidal movement. This may be indicative of an EM entering bed and may cause the state to transition from state 501 to state 502.

From state 502, motion may continue to be detected in bed, such as due to the EM rolling around, getting positioned, moving pillows, sheets, and/or blankets, reading a book, etc. State 502 may transition to state 503 while such motion continues to be detected. Alternatively, if motion is detected, then zero motion is detected, this may be indicative that state 505 has been entered by the monitored EM exiting bed. If this condition occurs, state 502 may transition to state 505, then back to state 501. Generally, state 504 may be interpreted as the EM being asleep and state 503 may be interpreted as the EM being awake. In some embodiments, more than a threshold amount of time (or some other form of determination that uses a form of threshold criterion at least partially based on time) in state 504 is necessary to classify the EM as asleep and more than a threshold amount of time (or some other form of determination that uses a form of threshold criterion at least partially based on time) in state 503 is necessary to classify the EM as awake. For instance, movement in bed of less than five seconds may be interpreted as the EM moving while still asleep if the EM was previously determined to be asleep. Therefore, if an EM transitions to state 503 from state 504, experiences some number of movement events, then returns to state 504 within less than a duration of time, the EM may be identified as having experienced a “sleep arousal” in which the EM's sleep is disturbed, but the EM has not been awoken. Such sleep arousals may be tracked together with or separate data may be maintained from episodes where the EM is judged to have fully awoken.

From state 503, the monitored EM may be determined to be exiting bed at state 505 and may become motionless at state 504. To be “motionless” at state 504 refers to no large movements being performed by the monitored EM, but the EM continuing to perform small motions due to vital signs. In some embodiments, only when the monitored EM's state is determined to be state 504 are vital signs treated as accurate and/or stored, recorded, or otherwise used to measure the EM's vital signs. Data collected during state 503 and state 504 may be used to determine the monitored EM's general sleep patterns (e.g., how much time tossing and turning, how much quality sleep, when deep sleep occurred, when REM sleep occurred, etc.). After an EM enters state 504 for a predefined period of time, the EM may be assumed to be asleep until the EM exits state 504. When an EM initially transitions to state 504, the EM may be required to stay in state 504 for some amount of time, such as two to five minutes, to be considered asleep. If an EM is in state 503 for at least a defined period of time, the EM may be identified as awake. However, if the EM enters state 503 from state 504 for less than the defined period of time, and returns to state 504, the EM may be identified as just moving within their sleep and has been continuously asleep.

FIG. 6 illustrates an embodiment 600 of beam-steering module 610 for targeting the direction in which fetal movement tracking (and possibly sleep tracking) is performed. Beam-steering module 610 can represent an embodiment of beam-steering module 230 of FIG. 2B. Generally, beam-steering module 610 may apply a weight to each antenna data stream received from radar subsystem 205, sum the weighted inputs, and output the combined weighted antenna data stream to radar processing module 210. The weights applied may introduce a delay to the input of a particular antenna, which can be realized by the weight being a complex value. By a delay being introduced to one or more of the antenna data streams received from the antennas, the antenna receive beam can be effectively steered.

In embodiment 600, three digital antenna data streams 620 (620-1, 620-2, 620-3) are received from radar subsystem 205 with each digital antenna data stream corresponding to a separate antenna. Therefore, in this embodiment, three antennas are present as part of radar subsystem 205. In other embodiments, radar subsystem 205 may have fewer (e.g., 2) or greater numbers (e.g., 4, 5, 6, 7, or more) of antennas, each with a corresponding raw antenna data stream output in digital form to beam-steering module 610.

Mixers 630 and combiner 640 can represent beam steering system 232. Each of antenna data streams 620 may be input to a separate mixer of mixers 630. Mixers 630 may be digitally implemented and may therefore represent software processes. Mixer 630-1 mixes antenna data stream 620-1 with a weight, represented by a complex value, output by channel weighting engine 231. Mixer 630-2 mixes antenna data stream 620-2 with a weight (which may be the same or differ from the weight applied at mixer 630-1), output by channel weighting engine 231. Mixer 630-3 mixes antenna data stream 620-3 with a weight (which may be the same or different from each of the weights applied at mixers 630-1 and 630-2), output by channel weighting engine 231.

Channel weighting engine 231, which can represent a software process, may perform a training process to determine the values (e.g., complex values) representative of the weights that should be output to each of mixers 630. In other embodiments, channel weighting engine 231 may be performed by separate specialized hardware or hardware that is incorporated as part of radar subsystem 205. The digital signals representing the weights output by channel weighting engine 231 may effectively apply a greater or smaller delay to each of antenna data streams 620. The weights applied via mixers 630 may be normalized to 1. Therefore, the sum of the three weights applied in embodiment 600 may sum to 1.

Beam steering system 232 and beam-steering module 610 can be used to implement weighted delay and sum (WDAS) beam-steering via mixers 630. Equation 1 details how WDAS can be implemented:

$$\hat{x} = {\sum }_{i = 1}^{M} {w}_{i} * {x}_{i} = {\sum }_{i = 1}^{M} ( {a}_{i} * {e}_{i}^{- j {πθ}_{i}} ) * {x}_{i} . \qquad Eq . 1$$

In Equation 1, wᵢ represents the channel weight, which can be a complex value to introduce phase delay; xᵢ represents the incoming digital radar data (e.g., a FMCW radar chirp) from radar subsystem 205; aᵢ represents the complex-valued weights that are responsible for phase-delaying different receive antenna signals with different magnitudes. The weightings output by channel weighting engine 231 may be determined by performing a least-squares optimization process. The least squares optimization process may be performed according to Equation 5.

$$minimize {‖ y - Xw ‖}^{2} \qquad Eq . 2$$

In Equation 2, y represents vectorized data generated using the target beam. X represents the antenna data stream data received from radar subsystem 205; w represents the weights that are to be learned by channel weighting engine 231. As part of a training process to determine the most effective weights to target the user, various weights may be tested (e.g., in a pattern, randomly) in an attempt to obtain a minimized output of Equation 2. For example, if enough randomized weights are tested, it can be expected that the minimized output value can be obtained within an amount of error. By minimizing the output value according to the least-squares optimization process, the weights corresponding to the beam direction that most closely targets where the user is located within the bed may be obtained. These weights may then be used for future monitoring of the user. Periodically or occasionally, retraining may be performed to compensate for the user moving within the bed and/or the orientation and/or location of the sleep detection device being changed.

Prior to use, weights may be determined offline to compensate for a known tilt of the radar subsystem, such as indicated in FIG. 4A and indicated by directions 450 and 452. When a user is present, the optimal direction is determined for the user, such as by sweeping or randomly selecting weights. When a user is not present, one or more directions to stationary objects that produce significant reflections can be determined such that these one or more directions can be avoided when targeting a user.

It should be understood that a learning process other than a least squares optimization process may be performed by channel weighting engine 231. For instance, in some embodiments, a user may assist in the training process by providing an input indicating a direction from the contactless sleep tracking device to where the user sleeps. In other embodiments, a different form of automated learning process may be performed to target the beam at the user.

Channel weighting engine 231 may be triggered to determine weights on system 200B being booted or turned on. If motion is detected by system 200B, such as via an on-board accelerometer, channel weights may be recalculated.

A summation of the weighted antenna data streams 635 (e.g., 635-1, 635-2, and 635-3), as output by mixers 630, may be received by combiner 640. Combiner 640 may output a single summed output 645 to radar processing module 210. By at least one weight (that causes a delay) applied by mixers 630 differing from the other weights applied by mixers 630, the beam is effectively steered in a direction, which may have a vertical and/or horizontal component. Processing by radar processing module 210 may be performed as detailed in relation to FIGS. 2A and 2B.

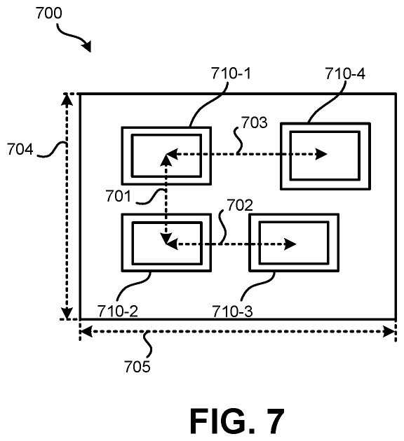

FIG. 7 illustrates an embodiment of a possible antenna layout of radar subsystem 700. Radar subsystem 700 may represent an embodiment of the integrated circuit that functions as radar subsystem 205. The entire IC may have dimensions of 6.5 mm (length 705) by 5 mm (width 704). In other embodiments, the entire IC has a length 705 by width 704 of between 7 mm by 7 mm and 4 mm by 4 mm. The illustrated embodiment of radar subsystem 205 has three receive antennas and one transmit antenna, but other embodiments may have a greater or fewer number of antennas. Radar subsystem 700 may have receive antennas 710-1, 710-2, and 710-3 distributed in an “L” pattern. That is, antennas 710-1 and 710-2 may be aligned on axis 701 and antennas 710-2 and 710-3 may be aligned on axis 702 which is perpendicular to axis 701, as illustrated in FIG. 7. The center of antenna 710-2 may be located 2.5 mm or less from the center of antenna 710-1. The center of antenna 710-2 may be located 2.5 mm or less from the center of antenna 710-3.

Transmit antenna 710-4 may be arranged separately from the L-shaped pattern of the receive antennas 710-1, 710-2, and 710-3. That is, in some embodiments, a center of transmit antenna 710-4 is not located on an axis with antenna 710-3 that is parallel to axis 701. In some embodiments, transmit antenna 710-4 is on axis 703 with center of antenna 710-1, with axis 703 being parallel to axis 702.

Each of antennas 710 may be hollow rectangular dielectric resonance antennas (DRAs). Each of antennas 710 may have a same set of dimensions. Alternatively, each of receive antennas 710-1, 710-2, and 710-3 may have the same dimensions and transmit antenna 710-4 may vary in dimensions from the receive antennas. In some embodiments, transmit antenna 710-4 has a larger width, such as 0.2 mm larger, than receive antennas 710-1, 710-2, and 710-3 but the same length.

In such an arrangement, the phase delay introduced by the applied weights between the antenna data stream of antenna 710-1 and the data stream of antenna 710-2 may affect the vertical direction of the receive beam and the phase delay introduced by weights between the antenna data stream of antenna 710-2 and data stream of antenna 710-3 may affect the horizontal direction of the receive beam (assuming the radar subsystem integrated circuit is present within the contactless sleep tracking device in approximately the same orientation).

In some embodiments, separate antennas are used for transmitting and receiving. For example, antenna 710-4 may be used exclusively for transmitting, while antennas 710-1, 710-2, and 710-3 are used exclusively for receiving.

Using a radar subsystem in which all the antennas are located on a single, relatively compact integrated circuit chip, as described, has been found to achieve a good balance of cost savings, reasonable ability to perform receive-side beam-steering, and a sufficiently wide antenna pattern in the horizontal plane that is able to encompass common bed sizes (e.g., queen, king, full, twin). At the same time, a device incorporating such a radar subsystem allows it to be placed sufficiently close to a bed (e.g., within 1 m) so that it can also function as a personal assistant, including alarm clock functionality (which can replace an alarm clock), a home control hub, and/or an entertainment touchscreen device.

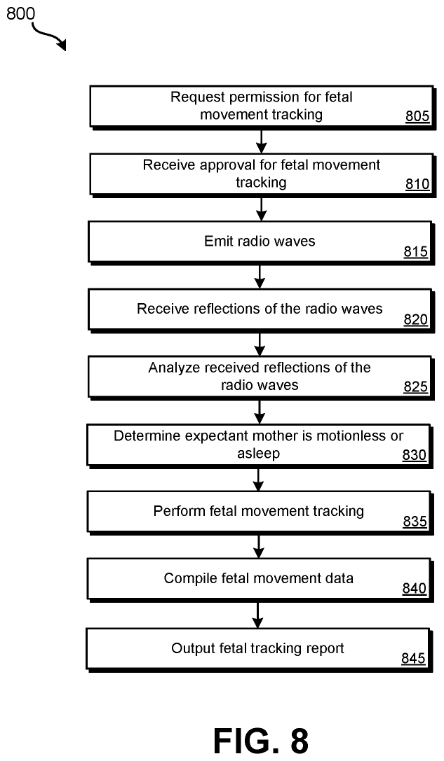

The devices, systems, and state machines of FIGS. 1-7 can be used to perform various methods. FIG. 8 illustrates an embodiment of a method 800 for performing contactless fetal movement tracking. Method 800 can be performed using system 100, or, more specifically FMTD 101 of FIG. 1.

At block 805, prior to any fetal tracking being performed and, potentially, prior to any radar being emitted, informed consent of the EM and, potentially, any other user may be required. Information about the data collected, how the data is stored, and when the conditions under which the data is deleted can be presented to the EM, such as via an electronic display, for acceptance. At block 810, approval can be received, such as by selection of a graphical element indicating “I agree.” If the EM does not consent to the term of use, no additional steps of method 800 are performed.

At block 815, radio waves are emitted. The radio waves emitted can be continuous-wave radar, such as FMCW. The raw waveform data passed to the radar processing module may include waveform data indicative of continuous sparse reflected chirps due to the radar subsystem operating in a continuous sparse sampling mode or due to the radar subsystem operating in a burst mode and a conversion process to simulate raw waveform data produced by the radar subsystem operating in a continuous sparse sampling mode being performed. The radio waves emitted at block 815 may be emitted in accordance with the FMCW radar scheme of FIG. 3. The radio waves emitted can be emitted by RF emitter 206 of radar subsystem 205.

At block 820, reflections of the radio waves may be received, such as by multiple antennas of RF receiver 207 of radar subsystem 205. The reflections received at block 820 may be reflected off of moving objects (e.g., a person having a heartbeat and breathing) and stationary objects. For each FMCW chirp emitted at block 815, a number of samples may be measured of reflected RF intensity, such as 64 samples, at block 820. Fewer or greater numbers of samples may be measured in other embodiments. A phase shift may be present in the radio waves reflected by a moving object.

At block 825, raw waveform data, which can also be referred to as raw chirp waterfall data, may be created based on received reflected radio waves by each antenna. The reflected radio waves may be indicative of distance and a phase shift. At a given frequency, such as 10 Hz, a number of samples may be taken, such as 64 samples. For each of these samples, intensity and phase shift data may be present, and may be output as a digital antenna data stream, with a separate antenna data stream being present for each antenna used to receive the reflected radio waves. Further processing may be performed in the digital domain. In other embodiments, the antenna data streams may be output by the radar subsystem as analog data and the weighting process may be performed in the analog domain. Over time, a window of raw waveform data may be created and stored in a buffer for analysis. Referring to FIG. 2, block 815 may be performed by radar processing module 210.

In some embodiments, method 800 can involve determining one or more weightings, as detailed in relation to FIGS. 2B and 6, to perform beam steering.

In some embodiments, at block 830, before any fetal movement is tracked, a determination may be made that the EM is present and still or is asleep. In some embodiments, this arrangement involves the state machine of FIG. 5 being assessed, possibly based on the output of an ML model that indicates sleep state. The EM may be required to be identified as in state 504. In some embodiments, the EM is required to be in state 504 for at least a predefined period of time, which can serve as a proxy indicating that the EM is likely asleep. In other embodiments, block 830 is not performed. That is, method 800 proceeds directly to block 835 from block 825.

At block 835, fetal movement tracking is performed. Fetal movement tracking can involve radar data being analyzed as detailed in relation to FIGS. 2A and 2B. Each instance of fetal movement that is identified (possibly while the EM is present and static or asleep) can be logged.

In embodiments where the EM's vital signs are monitored, a determination can be made as to the distance from the EM's vital signs that the fetus's movement can be expected to occur. Since FMCW is used, potentially in combination with beam steering, movement at particular distances in a particular direction can be monitored. Based upon the location of the EM's vital signs, the general location of the fetus can be determined. Fetal movement tracking can then be restricted to radar data gathered from this general location.

At block 840, as part of an ongoing process or after monitoring is complete, fetal movement data may be compiled. Determination of when fetal movement tracking is complete may be based on state machine 500. For example, when the EM is determined to be exiting bed at state 505 and/or a certain time of day has been passed, monitoring may be determined to be complete. As an example, if the EM exits bed before 5 AM, it may be assumed the EM is going to return to bed. However, if the EM exits bed after 6:30 AM, it may be assumed that the EM is done sleeping for the night and fetal monitoring is complete.

Compiling the fetal movement data can involve compiling data from the previous night (or, more generally, sleeping session), possibly along with data from a more extended period of time, such as the past 5 days, past week, past two weeks, past month, past two months, or some other duration of time longer than one day. Data that can be compiled include: number of fetal movements over the course of the night (or sleeping session); number of fetal movements per time period (e.g., per hour); average number of fetal movements per hour; comparison of the number of fetal movements over the course of the night with an average night; and/or an indication of a long-term trend (e.g., at least multiple days) of whether fetal movement is generally increasing, decreasing, or staying consistent.

At block 845, the fetal tracking report can be output for presentation to the EM. In some embodiments, the fetal tracking report is output for presentation by the FMTD on its display screen. Additionally or alternatively, the fetal tracking report may be output for presentation on another device, such as computerized device 180. Data included in the fetal tracking report can be stored by cloud-based server system 170 in association with a user account that was active on the FMTD during fetal movement tracking.

In order for the fetal tracking report to be output, a command (e.g., a spoken command) from the EM may need to be received by the FMTD that requests output of the fetal tracking report. In some embodiments, on determining that the EM is getting out of bed and, possibly, one or more conditions (e.g., it is morning or after a specific time), the fetal tracking report may be presented. Presentation of the fetal tracking report can include presentation of fetal movement data and/or fetal movement data being output using synthesized speech. If a separate computerized device is to be used to view the fetal tracking report, the EM (or another authorized user) can access the fetal tracking report by logging in the user account that was active at the FMTD when the fetal movement tracking was performed.

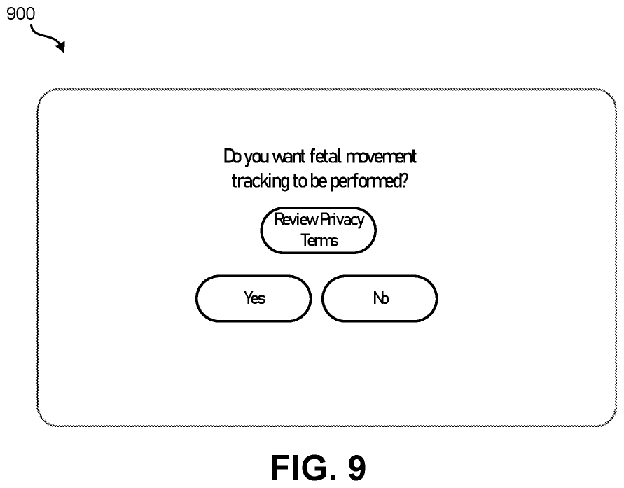

The FMTD, such as FMTD 101, can present various graphical user interfaces (GUIs) as part of the fetal movement tracking process. FIG. 9 illustrates an embodiment of a user interface 900 for indicating that a user desires to enable fetal movement tracking. The user, which would typically be an EM, can interact with various graphical elements to review the privacy data addressing how the data will be stored and used. The EM can also either assent or deny fetal tracking being performed.

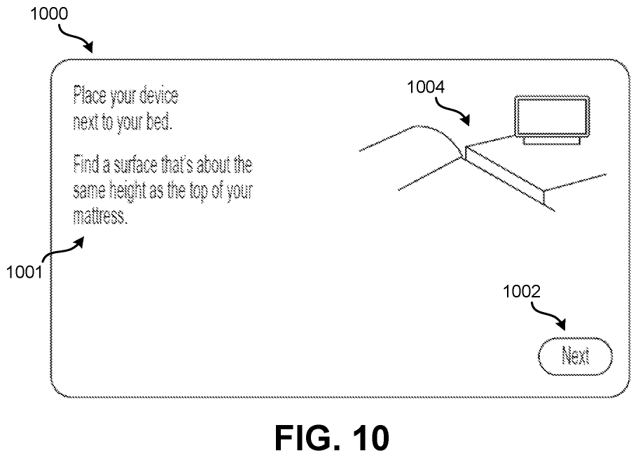

FIG. 10 illustrates an embodiment of a user interface 1000 for performing initial calibration for fetal movement tracking. Basic instructions on how to position the FMTD may be provided as instructions 1001. A diagram 1004 may illustrate how the FMTD should be positioned. The EM can then proceed to the next step by selecting graphical element 1002.

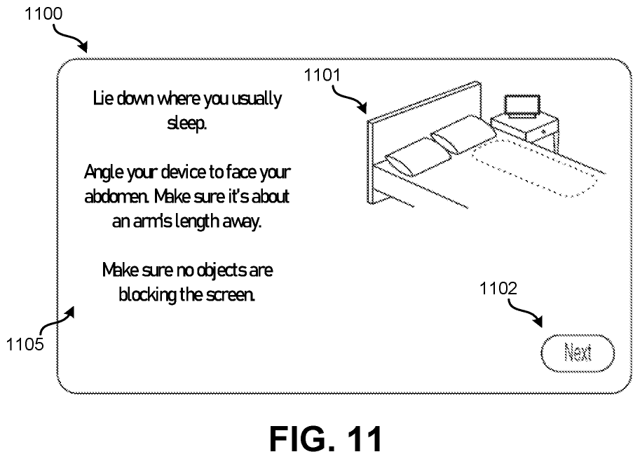

FIG. 11 illustrates an embodiment of user interface 1100 for performing initial set-up for fetal movement tracking. Basic instructions 1105 may be provided to help the EM orient the FMTD in relation to where she usually sleeps. Diagram 1101 may indicate a recommended sleeping arrangement. The EM can then proceed to the next step by selecting graphical element 1102.

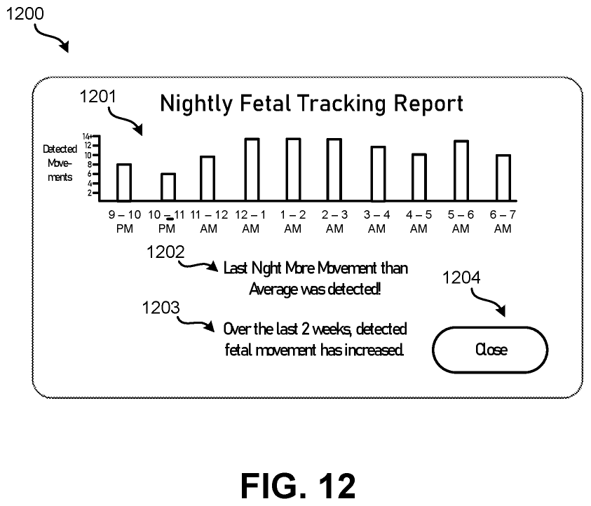

FIG. 12 illustrates an embodiment of a fetal movement tracking report 1200 as presented by an FMTD. In this example, graph 1201 is presented that breaks down the detected fetal movements by hour over the course of the night. Data compiled over a longer period of time than one night is presented as trend 1202 and trend 1203. Trend 1202 indicates a comparison between last night's detected movement and a longer-term average. Trend 1203 indicates that over the past two weeks, fetal movement has generally increased. If trend 1203 indicates that fetal movement has decreased, a recommendation may be presented that the EM contact her doctor.

Upon completion of review, the EM may select graphical element 1204. FMTD may then be used to perform other functions, such as the functions of a home assistant device, until fetal monitoring is to be performed again.

It should be noted that the methods, systems, and devices discussed above are intended merely to be examples. It must be stressed that various embodiments may omit, substitute, or add various procedures or components as appropriate. For instance, it should be appreciated that, in alternative embodiments, the methods may be performed in an order different from that described, and that various steps may be added, omitted, or combined. Also, features described with respect to certain embodiments may be combined in various other embodiments. Different aspects and elements of the embodiments may be combined in a similar manner. Also, it should be emphasized that technology evolves and, thus, many of the elements are examples and should not be interpreted to limit the scope of the invention.

Specific details are given in the description to provide a thorough understanding of the embodiments. However, it will be understood by one of ordinary skill in the art that the embodiments may be practiced without these specific details. For example, well-known, processes, structures, and techniques have been shown without unnecessary detail in order to avoid obscuring the embodiments. This description provides example embodiments only, and is not intended to limit the scope, applicability, or configuration of the invention. Rather, the preceding description of the embodiments will provide those skilled in the art with an enabling description for implementing embodiments of the invention. Various changes may be made in the function and arrangement of elements without departing from the spirit and scope of the invention.

Also, it is noted that the embodiments may be described as a process which is depicted as a flow diagram or block diagram. Although each may describe the operations as a sequential process, many of the operations can be performed in parallel or concurrently. In addition, the order of the operations may be rearranged. A process may have additional steps not included in the figure.

Having described several embodiments, it will be recognized by those of skill in the art that various modifications, alternative constructions, and equivalents may be used without departing from the spirit of the invention. For example, the above elements may merely be a component of a larger system, wherein other rules may take precedence over or otherwise modify the application of the invention. Also, a number of steps may be undertaken before, during, or after the above elements are considered. Accordingly, the above description should not be taken as limiting the scope of the invention.

What is claimed is:

1. A contactless fetal movement tracking device, comprising:
a housing;
a touchscreen electronic display housed by the housing;
a radar sensor housed by the housing, the radar sensor aimed such that when the housing is placed bedside, movement of an expectant mother within a bed is detected; and
a processing system housed by the housing, comprising one or more processors, that receives data from the radar sensor and the touchscreen electronic display, and outputs data to the touchscreen electronic display for presentation, wherein the processing system is configured to:
receive user input, via the touchscreen electronic display, requesting that contactless fetal movement monitoring be performed;
in response to the user input, perform a state analysis on the expectant mother to determine that the expectant mother is present and static in the bed;
while the state analysis indicates that the expectant mother is present and static in the bed, perform fetal movement tracking using radar data received from the radar sensor; and
cause a fetal tracking report to be presented by the touchscreen electronic display based on the performed fetal movement tracking.

2. The contactless fetal movement tracking device of claim 1, wherein the fetal tracking report is indicative of fetal movement occurring during a multi-hour period of time during a previous night while the expectant mother was asleep.

3. The contactless fetal movement tracking device of claim 1, wherein the performed fetal movement tracking using the radar data received from the radar sensor is at least in part performed using a trained machine learning model.

4. The contactless fetal movement tracking device of claim 1, wherein the performed fetal movement tracking using the radar data received from the radar sensor is performed by monitoring for movement a distance from a detected vital sign of the expectant mother.

5. The contactless fetal movement tracking device of claim 1, wherein the processing system is further configured to:
sort instances of detected fetal movement by time, wherein the fetal tracking report indicates a plurality of indications of movements per unit of time.

6. The contactless fetal movement tracking device of claim 1, further comprising:
a network interface that communicates with a remote server via the Internet, wherein data from the fetal tracking report is stored by the remote server in association with a user account linked to the expectant mother.

7. The contactless fetal movement tracking device of claim 1, wherein the state analysis comprises determining that the expectant mother is asleep based on the expectant mother having been present and static for at least a defined period of time.

8. The contactless fetal movement tracking device of claim 1, wherein the processing system is further configured to:
perform a comparison of the monitored fetal movement from a previous night with fetal movement data from an earlier time period, wherein
the fetal tracking report indicates a result of the performed comparison.

9. The contactless fetal movement tracking device of claim 1, wherein the processing system is further configured to:
calculate a trend over multiple days using the monitored fetal movement, the trend indicating whether fetal movement is generally increasing, decreasing, or staying constant over a time period comprising a plurality of nights, wherein
the fetal tracking report indicates the calculated trend.

10. The contactless fetal movement tracking device of claim 1, wherein the radar sensor emits frequency-modulated continuous wave radar.

11. The contactless fetal movement tracking device of claim 1, further comprising a beam steering module configured to electronically aim toward the expectant mother.

12. A method for performing contactless fetal movement tracking, the method comprising:
receiving user input, by a contactless fetal movement tracking device, requesting that contactless fetal movement monitoring be performed;
based at least in part on the user input, performing, by the contactless fetal movement tracking device, a state analysis on an expectant mother to determine that the expectant mother is present and static in a bed at which the contactless fetal movement tracking device is pointed;
performing, by the contactless fetal movement tracking device, fetal movement tracking using radar data received from a radar sensor of the contactless fetal movement tracking device while the state analysis indicates that the expectant mother is present and static in the bed; and
presenting, by the contactless fetal movement tracking device, on an electronic display, a fetal tracking report to based on the performed fetal movement tracking.

13. The method of claim 12, wherein the fetal tracking report is indicative of fetal movement occurring during a multi-hour period of time while the expectant mother was asleep.

14. The method of claim 12, wherein performing fetal movement tracking using the radar data received from the radar sensor comprises using a trained machine learning model.

15. The method of claim 12, wherein performing fetal movement tracking using the radar data received from the radar sensor comprises monitoring for movement a distance from a detected vital sign of the expectant mother.

16. The method of claim 12, further comprising:
sorting instances of detected fetal movement by time, wherein the fetal tracking report indicates a plurality of indications of movements per unit of time.

17. The method of claim 12, further comprising:
transmitting, via a network interface to a remote server, data included in the fetal tracking report; and
storing, by the remote server, the data in association with a user account linked to the expectant mother.

18. The method of claim 12, further comprising:
calculating a trend over multiple days using the monitored fetal movement, the trend indicating whether fetal movement is generally increasing, decreasing, or staying constant over a time period comprising a plurality of nights, wherein the fetal tracking report indicates the calculated trend.

19. The method of claim 12, further comprising:
receiving, via a microphone of the contactless fetal movement tracking device, a voice command requesting the fetal tracking report, wherein the fetal tracking report is presented in response to the received voice command.

20. A non-transitory processor-readable medium comprising processor-readable instructions configured to cause one or more processors of a contactless fetal movement tracking device to:
receive user input requesting that contactless fetal movement monitoring be performed;
based at least in part on the user input, perform a state analysis on an expectant mother to determine that the expectant mother is present and static in a bed;
perform fetal movement tracking using radar data received from a radar sensor incorporated as part of the contactless fetal movement tracking device, wherein the fetal movement tracking is performed while the state analysis indicates that the expectant mother is present and static in the bed; and
cause, on an electronic display of the contactless fetal movement tracking device, a fetal tracking report to based on the performed fetal movement tracking.
